(19) United States
(12) Patent Application Publication
(10) Pub. No.: US 2004/0024337 A1
(43) Pub. Date: Feb. 5, 2004

(54) Orthopedic casting material and the method of making the same

(75) Inventors: How Tseng, Taipei (TW); Jiunn-Liang Chen, San Chung City (TW); Ju-Yu Chueh, San Chung City (TW)

Correspondence Address: BACON & THOMAS, PLLC, 625 SLATERS LANE, FOURTH FLOOR, ALEXANDRIA, VA, 22314

(73) Assignee: BIOTECH ONE INC., Taipei (TW)

(21) Appl. No.: 10/255,573

(22) Filed: Sep. 27, 2002

(30) Foreign Application Priority Data
Aug. 5, 2002 (TW) 91117539

(51) Int. Cl. A61F 5/00

(52) U.S. Cl. 602/6

(57) ABSTRACT

An orthopedic casting material comprises a support layer made of thermoplastic material in the form of a sheet repeatedly formable for wrapping round a body member to be immobilized, and a buffer layer made of compressibly elastic thermoplastic material and bonded to one side of the support layer for buffering between the support layer and the body member to be immobilized.

### FIELD OF THE INVENTION

The present invention relates to an orthopedic casting material and, more particularly, to a light orthopedic casting material with sufficient strength and proper shape setting time and the method of making the same.

### BACKGROUND OF THE INVENTION

In early days, gypsum was used to make into plaster bandages for treatment of temporarily immobilizing the injured skeletons or directly treating them. Gypsum is a chalk like material containing calcium sulfate commonly made into two types: plaster cast and synthetic (or resin cast). The plaster bandages are to support and protect fractured bone, to prevent and correct malformation, or to immobilize injured tendon or ligament. However, these conventional plaster bandages have numerous drawbacks as outlined hereinafter

1. They are heavy and uncomfortable in use and may cause the patient unable to move freely.

2. The plaster cast plaster bandage requires a long drying time. The patient should be kept immovable for one to two days before the plaster cast solidified. Further, the plaster cast should be kept away from water. If wetted, the plaster cast will break easily. Further, because of poor ventilation capacity of the plaster cast, it tends to cause the patient itching and sensitizing.

3. The synthetic or resin cast plaster bandage is waterproof because it contains glass fibers. However, when a synthetic or resin cast is wetted, the sweltering inner cotton sleeve of the cast causes the patient severely uncomfortable.

4. During first week in therapy, the injured tissue of the patient normally will swell. The plaster bandage will compress the swelled tissue to cause pain to the patient and affect normal circulation of blood. When arrived the second week, the swelling of the injured tissue is reduced, at this time the plaster bandage become too loose to immobilize the injured portion. Because it is inconvenient to remove the plaster bandage from patient and to remold the plaster bandage again after it was solidified, the situations as described above sometime will make the patient causing other syndromes, such as compartment syndrome.

5. A power saw is used to remove the plaster bandage. The power saw may injure the patient accidentally when cutting the plaster bandage.

In order to eliminate the aforesaid drawbacks, different orthopedic splinting and casting materials and related fabrication methods have been developed.

Patent WO 00/57821 discloses an orthopedic splinting or casting material, which comprises three parts, namely, the flexible envelope, the filler, and the heating element. The flexible envelope is formed of a plastic bag. The filler is a thermoplastic compound of solid-liquid phase transfer temperature within 30° C.˜70° C. The heating element includes electrodes, and is adapted to heat the filler to its solid-liquid phase transfer temperature to make the filler soft and plastically deformable. The optimum heating temperature is within 40° C.˜60° C., preferably at 50° C. The drawback of this material is the restricted environment of use. Because the material contains electrodes for heating, an electric shock may occur when the user having a bath.

U.S. Pat. No. 4,661,535 discloses a thermoplastic composition, especially for use as an orthopedic splinting or casting material, which comprises crosslinked polymeric component and filler. The crosslinked polymeric component contains polycaprolactone and a thermoplastic rubber, such as styrene-butadiene-styrene and styrene-isoprene-styrene triblock copolymers, in a ratio of from about 4:1 to about 1.5:1 by weight. The crosslinking effect of the invention is carried out by peroxide and the filler is preferably a mixture of talc and silica. The ratio of polycaprolactone and thermoplastic rubber is from about 4:1 to about 1.5:1, a preferably ratio is from 2.5:1 to 1.5:1, such as 2:1. The ratio of polymeric component to filler is from about 5:1 to 1:1, a preferably ratio is from 3:1 to 2.1, such as 2.5:1. The composition further is added with titanium dioxide in an amount from 0.1% to 10% by weight of the composition, or preferably from 1% to 8%, such as 4% and the composition will become opacity. This composition requires too many components and complicated fabrication procedures so that the cost of this composition is very high.

U.S. Pat. No. 4,483,333 disclosed an orthopedic cast made of a mixture of polyethylene and thermoplastic polyester with a melting temperature between 50° C. and 100° C. The thermoplastic polyester can be polycaprolactone with an average molecular weight of over 5,000. In order to enhance the stability of the structure of the orthopedic cast and avoid it over-molding under molten status, the orthopedic cast is added fillers, such as silicon dioxide, mica and asbestos etc. Because the mixture is nonhomogenuous, polyethylene and thermoplastic polyester may separate to each other, therefore, if the mixture is heated to 60° C., a temperature higher than the melting temperature of the polycaprolactone but lower than the melting temperature of polyethylene, at this time, polycaprolactone will melt but polyethylene still is solid. Polycaprolactone now can be taken to be filler uniformly sprayed in polyethylene to increase the adhesive capacity of the mixture.

U.S. Pat. No. 5,897,513 disclosed an orthopedic splinting material with different colors, which comprises a glass fiber substrate and a hardenable resin. The glass fiber substrate is mainly constructed of glass fiber yarns with a plurality of individual colored filaments therein to make the orthopedic splinting material colorful. The glass fiber substrate is stretchable made from Raschel knit or Tricot knit. The hardenable resin can be isocyanate functional resin, such as polyurethane prepolymer. Additives, such as viscosity modifiers, UV stabilizers and antioxidants etc., may be added to the resin.

U.S. Pat. No. 5,713,835 disclosed an orthopaedic splinting or casting material, which comprises a substrate, agent and catalyst. The substrate is composed of a hardenable resin containing multi-functional vinyl ether monomer. The agent is to mix with the monomer to increases the viscosity thereof and the catalyst is capable of causing the resin to harden when exposed to actinic radiation. A suitable ultraviolet radiation includes visible light and UV light. The preferable wavelength of UV light is from 240 nm to 400 nm (preferably wavelength is 300 nm to 400 nm). The splinting or casting material may be exposed to the ultraviolet radiation for a period of from 5 to 10 minutes in order to cause the resin to harden.

The aforesaid prior art orthopedic casting/splinting materials and/or orthopedic material fabrication methods still have drawbacks in shape forming, manufacturing cost, convenience of use, and fabrication time.

### SUMMARY OF THE INVENTION

The primary object of the present invention to provide an orthopedic casting material fabrication method, which is practical of making an orthopedic casting material that has a thin thickness and sufficient strength to immobilize and protect the injured body member of the patient. It further comprises a buffer layer giving a wear comfort to the patient without interfering blood circulation in the injured body member of the patient.

The secondary object of the present invention to provide an orthopedic casting material that can be easily fastened to and removed from patient for enabling medical care persons to visually check the recovery of the injury.

The third object of the present invention to provide an orthopedic casting material fabrication method, which is practical of making an orthopedic casting material suitable for applying to various body portions.

The fourth object of the present invention to provide an orthopedic casting material, which has a proper solidifying time so that the medical care persons have sufficient time to immobilize the injured body member of the patient.

The fifth object of the present invention to provide an orthopedic casting material, which does not cause any side effects to the patient's skin.

The sixth object of the present invention to provide an orthopedic casting material, which has fair cost to reduce medicine cost, save medicine resource and reduce patient's expense.

The seventh object of the present invention to provide an orthopedic casting material, which has an easier way to manufacture it and the products made from the method have lower proportion of defect to meet the need of mass manufacture.

To achieve these objects of the present invention, an orthopedic casting material comprises a support layer made of thermoplastic material, which can be repeatedly formable by heating it. The support layer is for wrapping round patent's injured portion. A buffer layer made of flexible thermoplastic material is bonded to a side of the support layer for buffering between the support layer and the patient's body to be immobilized.

An orthopedic casting material fabrication method includes the steps of:

a) Mix thermoplastic resin and titanium dioxide and make the mixture into grains.

b) Well mix the grains with fatty polyester-based substrate and bridging agent into a mixture and then press the mixture into a sheet member with a thickness within 1 mm˜3 mm so as to obtain a support layer, and

c) Bond thermoplastic resin to a side of the support layer, so as to form a buffer layer thereon.

### BRIEF DESCRIPTION OF THE DRAWINGS

FIG. 1 is a perspective view of an orthopedic casting material constructed according to the present invention;

FIG. 2 is an applied view of the present invention before wrapping of the orthopedic casting material;

FIG. 3 is similar to FIG. 2 but showing the orthopedic casting material wrapped round the body member, and

FIG. 4 is a sectional view taken along line 4-4 of FIG. 3.

### DETAILED DESCRIPTION OF THE PREFERRED EMBODIMENT

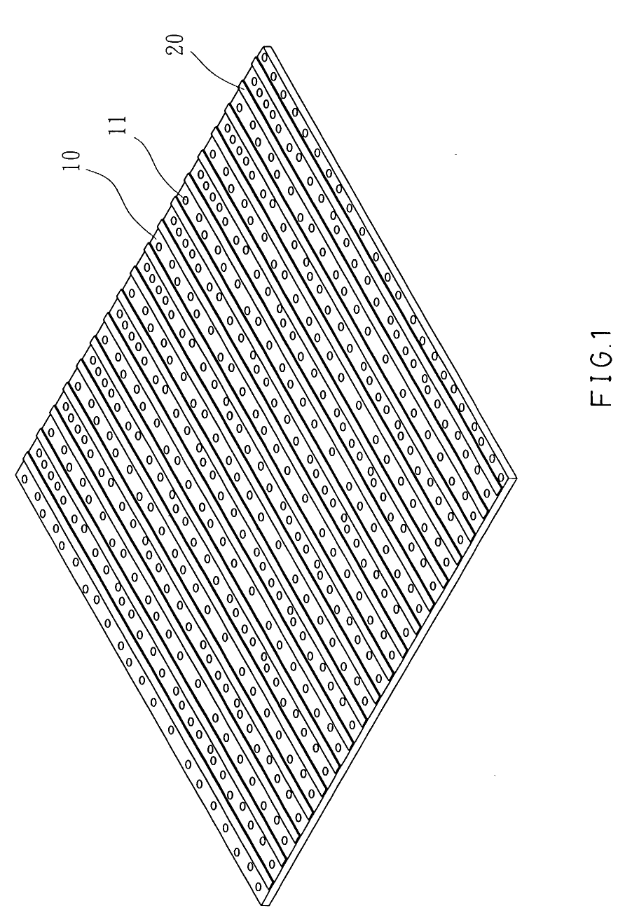

Referring to FIG. 1 an orthopedic casting material in accordance with the present invention is shown comprising a support layer 10 and a buffer layer 20.

The support layer 10 is made of flexible thermoplastic material in a sheet-like element. The support layer 10 is formable when heated for applying to the body member to be immobilized and is solidified when cooled for supporting and protecting patient's injured portion.

The buffer layer 20 is made of elastic thermoplastic material. The buffer layer 20 is fastened to a side of the support layer 10 for providing a buffering capacity between patient's body and the support layer 10.

According to the present invention, the support layer 10 is composed of fatty acid polyesters, such as poly lactic acid or polycaprolactone (PCL), as the base material and added with thermoplastic resin such as EVA (ethylene vinyl acetate), bridging agent, suitable amount of titanium dioxide and color powder. The support layer 10 preferably has a thickness within 1 mm˜3 mm, and the preferably thickness is 1.5 mm. The buffer layer 20 is made of EVA (ethylene vinyl acetate), which contains ethylene acetate 25%˜35% by weight, for example, 25%. The EVA for the buffer layer 20 contains ethylene acetate 35˜45% by weight, for example, 40%. EVA becomes softer when ethylene acetate content relatively increased. Therefore, the buffer layer 20 has a higher elastic buffering capacity. In order to provide more comfort to the user and to diminish the weight, the buffer layer 20 is bonded to the support layer 10 with gaps left therein. For example, the buffer layer 20 is bonded to the support layer 10 in the form of spots, strips, waves and meshes etc. According to the present preferred embodiment, the buffer layer 20 is arranged in the form of an array of stripes. Further, in order to increase the capacity of ventilation, the support layer 10 is punched to form air vents 11. The air vents 11 can be regularly or irregularly arranged in the support layer 10. The air vents 11 can be only on the support layer 10 or the can be on both of the support layer 10 as well as the buffer layer 20. According to the embodiment shown in the drawings, the air vents 11 formed in the support layer 10 only.

The method of manufacturing the orthopedic casting material of the present invention is outlined hereinafter.

The orthopedic casting material according to the present invention uses polyesters as base material and is added with thermoplastic resin to improve material viscosity, bridging agent to improve compatibility between polyesters and thermoplastic resin, and a small amount of titanium dioxide and color powder to change material color. The fabrication method includes the steps of:

A. Compound thermoplastic resin and titanium dioxide respectively;

B. Mix the compounded thermoplastic resin and titanium dioxide with polyester substrate, bridging agent and color powder and put the mixture in a dual screw extruding machine to press it into a sheet member with uniform thickness, and

C. Bond a layer of thermoplastic resin to a side of the sheet member when the sheet member delivered out of the extruding die of the extruding machine and pouch it.

The ratio of poly-L-lactic acid to EVA is about 4:1, preferably is 2.1, or most preferably is 1.8:1. The content of bridging agent is in an amount from 5% to 15% by weight of the composition of poly-L-lactic acid and EVA, preferably is 10%. The content of titanium dioxide is in an amount from 0.05% to 3% by weight of the composition of poly-L-lactic acid and EVA and bridging agent, or preferably in the amount of 0.125%. The thickness of the support layer is controlled within 1 mm˜3 mm to achieve optimum mechanical strength and plasticizing time. Most preferably, the thickness of the support layer is controlled at 1.5 mm. After bonding of molten EVA to the support layer, a buffer layer is formed, providing high viscosity for convenient application.

During fabrication, thermoplastic resin and titanium dioxide are mixed at first for the purpose that the finished product has all material properties of polyester substrate, for example, high strength. In other words, when using thermoplastic resin and titanium dioxide to modify material quality, it does not affect the strength of the material. The final product maintains the advantages of every material used.

Polyesters for the substrate can be selected from poly lactic acid, polycaprolactone. Thermoplastic resin can be obtained from EVA. Because EVA becomes softer when ethylene acetate content relatively increased. Therefore, different compositions of EVA are selected to achieve the aforesaid orthopedic casting material. According to this invention, two EVA of different ethylene acetate content, i.e., 28% and 40% are selected. EVA containing 28% ethylene acetate is adapted to mix with selected substrate. EVA containing 40% ethylene acetate is adapted to be bonded to the extruded product. A material made according to the present invention facilitates final fixation by the medical care person, does not absorb water, and is waterproof. Therefore, this material has little limitation in application.

According to tests, only a small amount of titanium dioxide and color powder can be added to the composition. Excessive amount of titanium dioxide and color powder severely lowers the strength of the material. The problem becomes more serious when the ratio of titanium dioxide is relatively increased. According to tests, the most optimum content of titanium dioxide is in the amount 0.125% by weight of the composition of poly-L-lactic acid and EVA and bridging agent.

The invention also enhances ventilation and sweat draining functions. The key point to achieve these functions is the bonding of an EVA compound containing 40% ethylene acetate content to the sheet member extruded from the extruding machine and the punching of air vents in the material. During bonding, strips of EVA are adhered to the extruded sheet member in proper order at a predetermined pitch. This bonding method reduces contact area between the orthopedic casting material and the body member, providing good ventilation.

### EXAMPLE

Mix 320 g EVA containing 28% ethylene lactate with 1.25 g titanium dioxide into grains. Then, mix the grains thus obtained with 580 g poly-L-lactic acid and 250 g bridging agent and 1.75 g green color powder and 0.75 g yellow color powder in a dual screw extruding machine, enabling the mixture to be extruded into a sheet member having the thickness of 1.5˜2 mm by the extruding machine. During extruding process, the dual screw extruding machine is controlled at temperatures 170° C., 170° C., 180° C., and 190° C. at four stages respectively, and the speed of the screws is controlled at 90 r.p.m. And then, rapidly adhere EVA strips containing 40% ethylene acetate to the extruded warm sheet member coming out of the extruding die of the extruding machine. At final, the finished product is cooled down to room temperature.

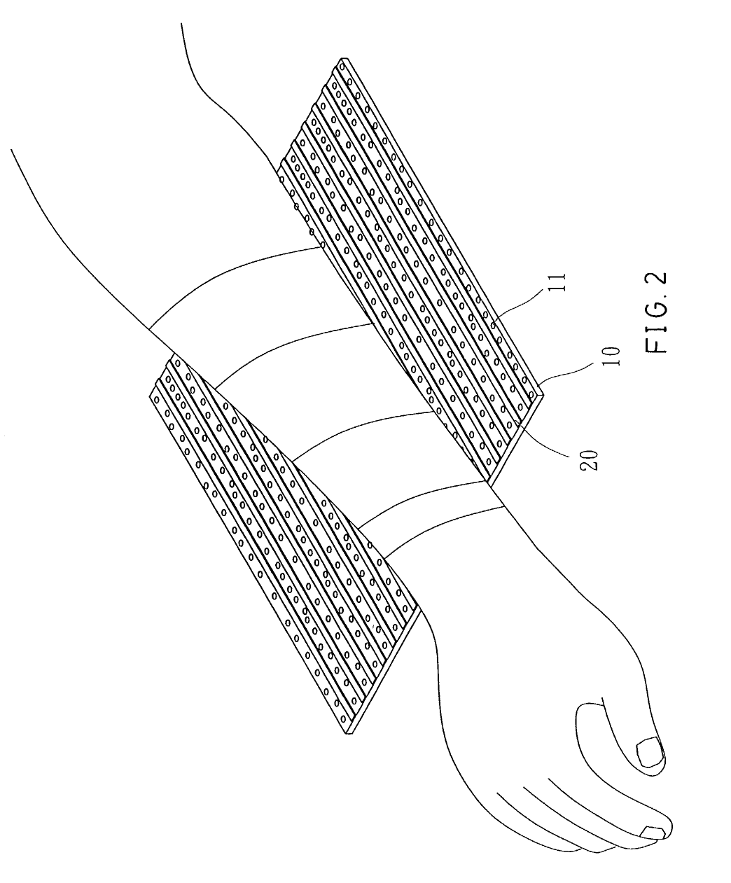

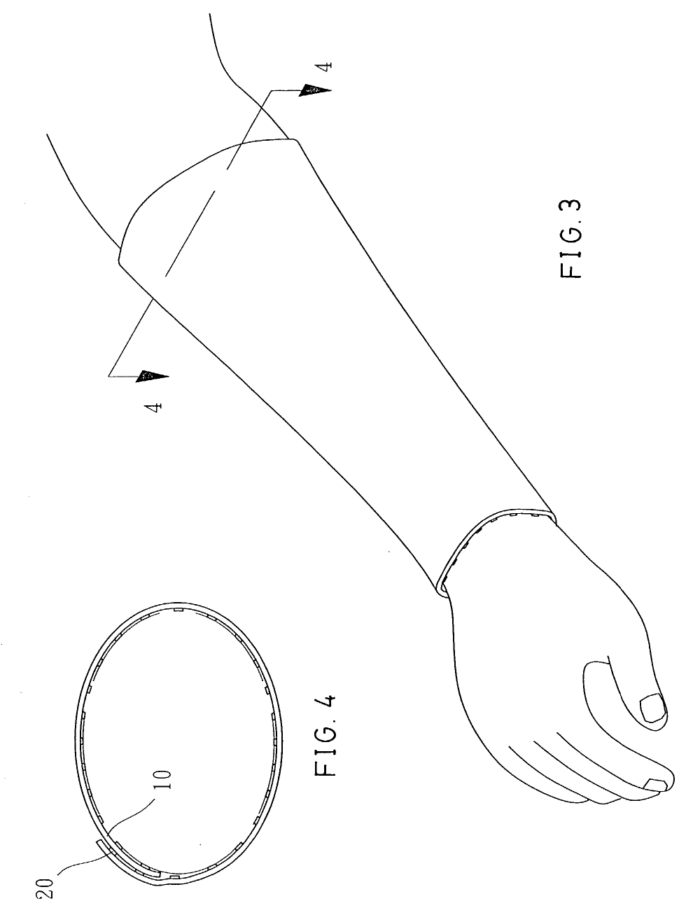

The use of the finished product thus obtained is described hereinafter. At fist, heat a suitable size of the orthopedic casting material to at least 65° C., or preferably 75˜90° C. The heating time is subject to the temperature desired. The heating can be achieve by dipping the material in hot water, baking the material in a baking stove, or using a hot air blower to blow hot air to the material. When heated to the desired temperature, the orthopedic casting material is wrapped round the body member to be immobilized as shown in FIGS. 2 and 3. At this time, the overlapped area of the buffer layer 20 is adhered to the support layer 10. The shape of the orthopedic casting material is set about 1˜2 minutes after wrapping. The shape setting temperature of the orthopedic casting material is at about 60˜65° C. This application procedure is simple and easy.

As indicated above, the buffer layer has open spaces in it and keeps the support structure (i.e., the support layer) from direct contact with the body member. Because the buffer layer is soft and provides good ventilation, the orthopedic casting material gives a comfort to the patient. Because the buffer layer is compressibly elastic, it gives little pressure to the circulation of blood in the injured body member when the injured body member begun to swell at the initial stage. Therefore, the patient does not feel much pain when the injured body member immobilized by the orthopedic casting material. When checking the injury or rewrapping the orthopedic casting material, use a hot air blower to heat the overlapped area of the orthopedic casting material, enabling the overlapped area of the orthopedic casting material to be opened. When rewrapped, the overlapped are is heated to a softened status and bonded together again. Therefore, the orthopedic casting material is easy to apply and, can be repeatedly used.

What the invention claimed is:

1. An orthopedic casting material, comprising:
a support layer made of flexible thermoplastic material in the form of a sheet for wrapping round a patient's injured portion to be immobilized, and
a buffer layer made of elastic thermoplastic material and provided at one side of said support layer for buffering between said support layer and the patient's injured portion.

2. The orthopedic casting material as claimed in claim 1, wherein said buffer layer is arranged on a side of said support layer and has gaps therein.

3. The orthopedic casting material as claimed in claim 2, wherein said buffer layer is bonded to said support layer in the form of spaced strips.

4. The orthopedic casting material as claimed in claim 2, wherein said buffer layer is bonded to said support layer in the form of spots.

5. The orthopedic casting material as claimed in claim 2, wherein said buffer layer is bonded to said support layer in the form of meshes.

6. The orthopedic casting material as claimed in claim 2, wherein said buffer layer is bonded to said support layer in the form of spaced waves.

7. The orthopedic casting material as claimed in claim 1, wherein said support layer has air vents.

8. The orthopedic casting material as claimed in claim 7, wherein said buffer layer has air vents in communication with the air vents of said support layer.

9. The orthopedic casting material as claimed in claim 1, wherein said support layer is repeatedly formable when heated to over 65° C., and the shape setting temperature of the orthopedic casting material is at about 60˜65° C.

10. The orthopedic casting material as claimed in claim 9, wherein said support layer comprises a fatty polyester-based substrate, thermoplastic resin, bridging agent, and titanium dioxide.

11. The orthopedic casting material as claimed in claim 10, wherein the material for said substrate is selected from the material group of poly lactic acid and polycaprolactone.

12. The orthopedic casting material as claimed in claim 10, wherein said thermoplastic resin is ethylene vinyl acetate.

13. The orthopedic casting material as claimed in claim 1, therein said support layer has a thickness within 1˜3 mm.

14. The orthopedic casting material as claimed in claim 12, wherein said ethylene vinyl acetate contains ethylene acetate 25˜35% by weight.

15. The orthopedic casting material as claimed in claim 1, wherein said buffer layer is made of ethylene vinyl acetate containing ethylene acetate 35˜45% by weight.

16. An method of fabricating an orthopedic casting material, comprising the steps of:
(a) compounding thermoplastic resin and titanium dioxide and making them into grains;
(b) mixing said grains with fatty polyester-based substrate and bridging agent into a mixture and then pressing said mixture into a sheet member with a thickness within 1˜3 mm so as to obtain a support layer, and
(c) bonding thermoplastic resin to a side of said support layer, so as to form a buffer layer at the side of said support layer.

17. The orthopedic casting material fabrication method as claimed in claim 16, wherein said buffer layer is bonded to said support layer with gaps left in said buffer layer.

18. The orthopedic casting material fabrication method as claimed in claim 17, wherein said buffer layer is extruded from a thermoplastic resin through an extruding machine in the form of strips and then bonded to said support layer at a pitch before shape setting of said support layer.

19. The orthopedic casting material fabrication method as claimed in claim 16, further comprising a step of punching the orthopedic casting material to make air vents thereon after step (c).

20. The orthopedic casting material fabrication method as claimed in claim 16, wherein the thermoplastic material used in step (a) is ethylene vinyl acetate containing ethylene acetate 25˜35% by weight; the material for the fatty polyester-based substrate used in step (b) is selected from the material group of poly lactic acid and polycaprolactone; the thermoplastic material used in step (c) is ethylene vinyl acetate containing ethylene acetate 35˜45% by weight.
